(19) United States
(12) Patent Application Publication
(10) Pub. No.: US 2023/0285721 A1
(43) Pub. Date: Sep. 14, 2023

(54) DEVICES, SYSTEMS, AND METHODS FOR CATHETER WITH ACTUATING PORTION

(71) Applicant: Boston Scientific Medical Device Limited, Galway (IE)

(72) Inventors: Amit Bharos, Jabalpur (IN); Swami Upadhyay, Raipur (IN); Kanishk Chaudhary, Anand (IN)

(73) Assignee: Boston Scientific Medical Device Limited, Galway (IE)

(21) Appl. No.: 18/119,681

(22) Filed: Mar. 9, 2023

Related U.S. Application Data
(60) Provisional application No. 63/318,136, filed on Mar. 9, 2022.

(51) Int. Cl. A61M 25/01; A61M 25/00

(52) U.S. Cl. CPC A61M 25/0147; A61M 25/0029; A61M 25/003

(57) ABSTRACT

The disclosure relates generally to the field of medical devices for accessing body lumens. In an embodiment, a catheter may comprise a shaft comprising a proximal end, a distal end, an actuating portion proximal to the distal end, and a first lumen extending along a length of the shaft between the proximal and distal ends. A plurality of slots may be disposed and spaced-apart from each other along the actuating portion. The plurality of slots may extend radially into the first lumen.

### CROSS REFERENCE TO RELATED APPLICATIONS

This application claims the benefit of U.S. Provisional Patent Application Ser. No. 63/318,136 filed on Mar. 9, 2022, the disclosure of which is incorporated herein by reference.

### FIELD

The present disclosure relates generally to the field of medical devices for accessing a body lumen. In particular, the present disclosure relates to medical devices, systems, and methods for targeted access to one or more body lumens, e.g., via a catheter with an actuating portion.

### BACKGROUND

Medical professionals may face technical challenges when accessing a body lumen, such as when performing endoscopic cannulation procedures that may involve advancing a guidewire and/or endoscopic device (e.g., sphincterotome, cannula, catheter, or the like) against, into, or through tortuous patient anatomies. For example, a target body lumen, e.g., a biliary sphincter, may be oriented at a difficult angle relative to the endoscopic device, have a very small or sealed opening, include a tortuous anatomy, or have blockages formed, e.g., stones, or benign or malignant strictures. Precise control of movement and force of the device and guidewire can be challenging. Even experienced medical professionals may require multiple attempts to achieve successful opening and access to body lumens, especially when working against frictional and patient-specific pathologies of a specific body lumen. The likelihood of causing trauma to tissues comprising or surrounding a target body passageway may increase with the number of opening or entry attempts. In some instances, the medical professional may abort the procedure entirely. In other instances, traumatized tissue may be prone to post-operative inflammation. It is with these considerations in mind that the improvements of the present disclosure may be useful.

### SUMMARY

In one aspect of the present disclosure, a catheter may comprise a shaft comprising a proximal end, a distal end, an actuating portion proximal to the distal end, and a first lumen extending along a length of the shaft between the proximal and distal ends. A plurality of slots may be disposed and spaced-apart from each other along the actuating portion, the plurality of slots extending radially into the first lumen.

In various embodiments described herein and otherwise within the scope of the disclosure a wire may be coupled to the distal end of the shaft. The wire may extend externally along the actuating portion of the shaft. The wire may be disposed within the first lumen proximal to the actuating portion. The wire may be disposed substantially at a midpoint across the plurality of slots. Each of the plurality of slots may extend radially through the first lumen. A second lumen may extend through the shaft, wherein the plurality of slots is external to the second lumen. A wall thickness about the second lumen may be approximately 0.075 mm or about 0.05 mm to about 0.10 mm. A cross-sectional profile of the plurality of slots transverse to a longitudinal axis along the length of the shaft may comprise a U-shape, a V-shape, a box-shape, or a combination thereof. An axial spacing between each of the plurality of slots along the length of the shaft may be longer than an axial length of each of the plurality of slots. The plurality of slots may be uniformly distributed along a length of the actuating portion. A handle may be coupled to a proximal end of the wire. The handle may be configured to proximally and distally translate the wire through the first lumen.

In another aspect of the present disclosure, a catheter may comprise a shaft comprising a proximal end, a distal end, a central longitudinal axis therethrough, an actuating portion proximal to the distal end, and a first lumen extending along a length of the shaft between the proximal and distal ends. A wire may be coupled to the distal end. The wire may extend external to the shaft along the actuating portion. The wire may extend within the first lumen proximal to the actuating portion. At least one slot may be disposed along the actuating portion. The slot may extend radially into the first lumen. The slot may be disposed between the central longitudinal axis and the wire.

In various devices embodiments described herein and otherwise within the scope of the disclosure, the slot may extend radially through the first lumen. A second lumen may extend along the length of the shaft. The slot may be external to the second lumen. A wall thickness of the second lumen may be approximately 0.075 mm or between about 0.05 mm to about 0.10 mm.

In another aspect, a method of cannulating an opening in a body lumen may comprise inserting a catheter into the body lumen of a patient. A wire coupled to a distal end of a catheter may be proximally translated. A portion of the catheter may be curved towards the opening in the body lumen. The wire may extend through at least a portion of a first lumen of the catheter. The catheter may comprise a plurality of slots along the actuating portion of the catheter, the plurality of slots extending radially into the first lumen.

In various embodiments of devices and methods described herein and otherwise within the scope of the present disclosure curving the actuating portion of the catheter may comprise reducing an axial width of each of the plurality of slots. A fluid may be supplied through a second lumen of the catheter into the body lumen. Proximally translating the wire may comprise pulling the wire toward a handle of the catheter external to the plurality of slots along the actuating portion. A portion of the body lumen may be cut by electrically activating the wire.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1A illustrates a perspective view of a catheter in a bowed configuration, according to an embodiment of the present disclosure.

FIG. 1B illustrates a perspective view of an exemplary embodiment of a handle of a catheter, such as for use with the catheter of FIG. 1A.

FIG. 2 illustrates a perspective view of a catheter in a bowed configuration, according to an embodiment of the present disclosure.

FIG. 3A illustrates an isometric view of a catheter, according to an embodiment of the present disclosure.

FIG. 3B illustrates a right view of the catheter of FIG. 3A.

FIG. 3C illustrates a perspective view of the catheter of FIGS. 3A and 3B in a bowed configuration.

FIG. 3D illustrates a cross-section of the catheter of FIGS. 3A-3C.

FIG. 3E illustrates another cross-section of the catheter of FIGS. 3A-3D.

FIG. 4A illustrates a right view of a slot of a catheter, according to an embodiment of the present disclosure.

FIG. 4B illustrates a right view of a slot of a catheter, according to an embodiment of the present disclosure.

FIG. 4C illustrates a right view of a slot of a catheter, according to an embodiment of the present disclosure.

FIG. 5A illustrates an isometric view of a finite element analysis of a catheter comprising slots, according to an embodiment of the present disclosure.

FIG. 5B illustrates an isometric view of a finite element analysis of a catheter, according to an embodiment of the present disclosure.

### DETAILED DESCRIPTION

The present disclosure is not limited to the particular embodiments described. The terminology used herein is for the purpose of describing particular embodiments only and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure may be described with specific reference to medical devices and systems (e.g., endoscopic devices, accessory tools, and/or guidewires inserted near or through a papilla, or the like) for selective access to, aligning with, cannulation, enlarging, and/or cutting of, the opening to the common bile duct (CBD) or pancreatic duct (PD) during endoscopic retrograde cholangiopancreatography (ERCP), it should be appreciated that such medical devices and systems may be used in a variety of medical procedures for navigating one or more devices through ductal, luminal, vascular, or body lumen anatomies, including, for example, interventional radiology procedures, balloon angioplasty/angiography procedures, thrombolysis procedures, urological or gynecological procedures, and the like. The medical devices herein are also not limited to use with duodenoscopes and may include a variety of medical devices for accessing body passageways, including, for example, catheters, ureteroscopes, bronchoscopes, colonoscopes, arthroscopes, cystoscopes, hysteroscopes, and the like. The disclosed medical devices and systems may also be inserted via different access points and approaches, e.g., percutaneously, endoscopically, laparoscopically, or combinations thereof.

As used herein, the singular forms “a,” “an,” and “the” are intended to include the plural forms as well, unless the context clearly indicates otherwise. The terms “comprises” and/or “comprising,” or “includes” and/or “including” when used herein, specify the presence of stated features, regions, steps, elements and/or components, but do not preclude the presence or addition of one or more other features, regions, integers, steps, operations, elements, components and/or groups thereof.

As used herein, “proximal end” refers to the end of a device that lies closest to the medical professional along the device when introducing the device into a patient, and “distal end” refers to the end of a device or object that lies furthest from the medical professional along the device during implantation, positioning, navigation, or delivery.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. The recitation of numerical ranges by endpoints includes all numbers within that range, including the endpoints (e.g., 1 to 5 includes 1, 1.5, 2, 2.75, 3, 3.80, 4, and 5).

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

To facilitate smooth and efficient entry of a catheter into/through a target body lumen, medical professionals may manually rotate, oscillate, linearly advance, and/or reciprocate the catheter to “wiggle” against, into, or through the body lumen. For example, ERCP may be performed by gastroenterologists and surgical endoscopists to diagnose and treat various disorders of the pancreaticobiliary system. Physicians in such procedures may use a catheter such as a sphincterotome to cannulate an opening in a body lumen (e.g., the papillary orifice, the CBD, the PD, or the like). A catheter may include a guidewire lumen, so that after initial cannulation the guidewire can be inserted through the guidewire lumen and into the opening of the body lumen, e.g., the CBD or PD. A catheter may include a cutting wire for enlarging/cutting/accessing tissue to enlarge the opening for access (e.g., a sphincterotomy or the like). Alternatively, the guidewire may be inserted ahead of the catheter and then used to guide the catheter to and into the opening, e.g., for enlarging in the case of a sphincterotome. A catheter may have a flexible distal end with an actuating portion that may be articulated (e.g., bowed, bent, steered, manipulated, or the like) by articulating a wire (e.g., a cutting wire) of the device. Bowing of a catheter may be performed to facilitate cannulation of the opening of the body lumen, e.g., inserting the catheter through the papilla and into or towards the hepatopancreatic duct or common bile duct. The distal end of the catheter may need to be extended into the opening of the body lumen, so that the cutting wire may extend to the tissue of the opening for enlarging or cutting. The target tissue or lumen to be treated may be located further through the opening of the body lumen, past the point of initial cannulation, so that the device is further steered through the body lumen and opening to the target tissue.

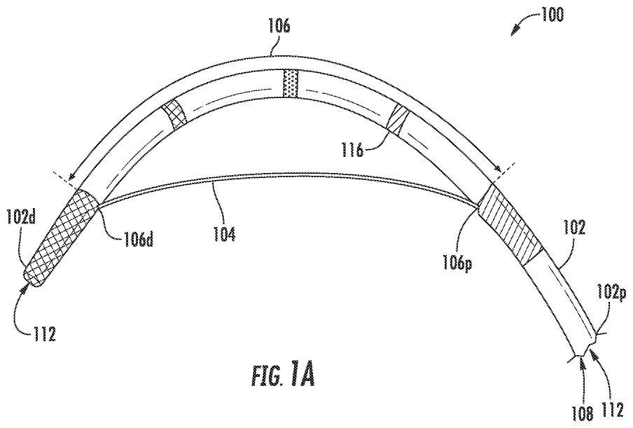

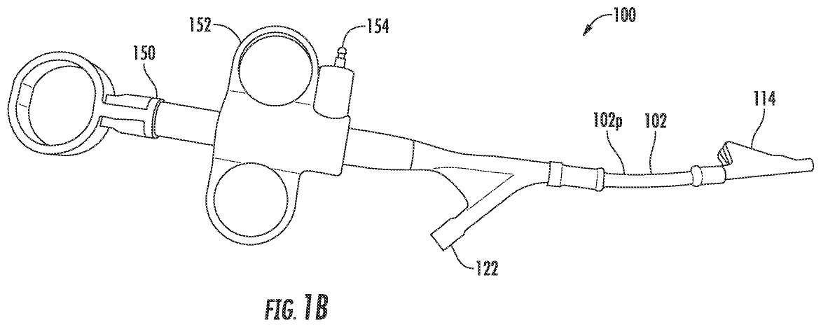

Referring to FIG. 1A, a perspective view of a catheter 100 is illustrated, according to an embodiment of the present disclosure. The catheter 100 includes a shaft 102 having a proximal end 102p and a distal end 102d. The shaft 102 includes an actuating portion 106 that can be manipulated substantially out of alignment with a remainder of the shaft 102 extending from the actuating portion 106 to the proximal end 102p. The actuating portion 106 can be manipulated, e.g., to direct the distal end 102d toward a target area, such as an opening of a body lumen, e.g., to direct an accessory and/or fluids (e.g., contrast fluid) extending through an accessory lumen 112 of the shaft 102 to the body lumen. The catheter 100 is in a bowed configuration with the actuating portion 106 being pulled by a wire 104 into an arc. The wire 104 extends through a wire lumen 108 along the shaft 102 proximal of the actuating portion 106. The wire 104 extends externally outside of the shaft 102 along the actuating portion 106 from a proximal end 106p of the actuating portion 106 to where the wire 104 is attached to a distal end 106d of the actuating portion 106. The actuating portion 106 may transition between the bowed configuration and a substantially straight configuration by translating the wire 104 through the wire lumen 108 such that tension is applied or released from the distal end 106d of the actuating portion 106. One or more markings 116 along the shaft 102, e.g., as illustrated, along the actuating portion 106 and also bordering the actuating portion 106, including the distal end 102d, may assist with visualizing portions of the shaft 102 during navigation and manipulation of the catheter 100.

Referring to FIG. 1B, a proximal portion of a catheter, such as the catheter 100 of FIG. 1A, is illustrated, according to an embodiment of the present disclosure. The proximal end 102p of the shaft 102 of the catheter 100 of FIG. 1A is coupled to a handle 150 in FIG. 1B. The handle 150 includes a slider 152 that is movable along the handle 150. The slider 152 is coupled to the wire 104 of FIG. 1A, and the slider 152 may be moved to translate the wire 104 and manipulate the actuating portion 106 of the catheter 100. An electrical connector 154 is disposed on the slider 152 that may electrically couple the wire 104 to a power source for electrically activating the wire 104 for a medical procedure as described herein. The accessory lumen 112 is in fluid communication with a first port 114 for introducing and removing an accessory into the accessory lumen 112. A second port 122 may be in fluid communication with an additional lumen of the shaft 102 for an additional accessory or fluid, e.g., a contrast fluid.

Referring to FIG. 2, a perspective view of a catheter 200 in a bowed configuration, according to an embodiment of the present disclosure, is illustrated. A distal end 202d of a shaft 202 of the catheter 200 is being manipulated, e.g., towards a body lumen opening. An accessory 240 may be delivered through an accessory lumen 212 of the shaft and/or the shaft 202 may be translated along the accessory 202. An actuating portion 206 of the shaft 202 is arced in the bowed configuration. The actuating portion 206 is being pulled by a wire 204 into the bowed configuration. The wire 204 extends through a wire lumen 208 along the shaft 202 proximal of the actuating portion 206, and outside of the shaft 202 along the actuating portion 206 from a proximal end 206p to a distal end 206d of the actuating portion 206. The actuating portion 206 may transition between a bowed configuration and a substantially straight configuration by translating the wire 204 through the wire lumen 208 such that tension is applied or released from the distal end 206d of the actuating portion 206. A kink 218, as will be further defined and described below, has developed along the actuating portion 206. The kink 218 along the actuating portion 206 may cause the bowed actuating portion 206 to have a less smooth and harsher transition along the arc of the actuating portion compared to, e.g., the arc of the actuating portion 106 in the bowed configuration of FIG. 1A or other bowed configurations illustrated and described herein.

As used herein, “kink”, “kinked”, and “kinking” refers to a sharp curve along a bow, bend, arc, or twist. A kink may occur along a length of a shaft when the shaft is elastically or plastically deformed an extraneous amount of distance per axial length or deformed an extraneous number of times such that a portion of the shaft along an inside of an arc undergoes excessive compression. A kink could cause the shaft of a device to decrease in performance or fail. For example, a kink may disrupt the integrity of an outer surface of a shaft such that one or more lumens within the shaft collapses, is punctured, and/or is occluded. A kink may disrupt an actuating portion of a shaft from further forming a desirable arc for a procedure in a bowed configuration. A kink may result in a fold at a localized area of a shaft, which may be at about a center point along an arc of a bowed curve. A kink could block or shut off access through the first and/or second lumens, compromising, e.g., the access of tools or flow of fluid therethrough.

The ability for an actuating portion of a shaft of a catheter to reversibly transition to a bowed configuration may be important for a medical procedure. Desirable bowing characteristics of a shaft of a catheter, e.g., for rapid cannulation, may include: a uniformly smooth bowing angle arc, ease of transitioning to the bowed configuration with minimal pull force on a wire, and stability of the actuating portion while in the bowed configuration. Promoting these bowing characteristics may reduce trauma to body lumens and may reduce time of a procedure while the patient may be sedated, experiencing trauma caused by medical devices of the procedure, and/or exposed to x-ray or other visual assistance. Many of these desirable bowing characteristics of an actuating portion of a shaft may be improved as a length of the actuating portion is increased. For example, an actuating portion length of about 30 mm may have better bowing performance than an actuating portion length of about 20 mm. However, increasing the length of the actuating portion may also require increasing a length of the wire that is external to the shaft, which may undesirably cause a deeper cutting of tissue when the shaft is in a bowed configuration and the wire is electrically activated. Therefore, it may be desirable to improve bowing performance of an actuating portion without extending a length of the actuating portion.

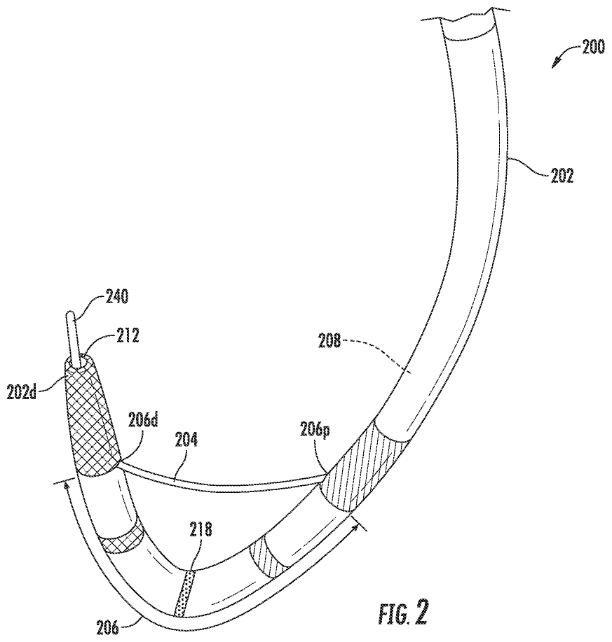

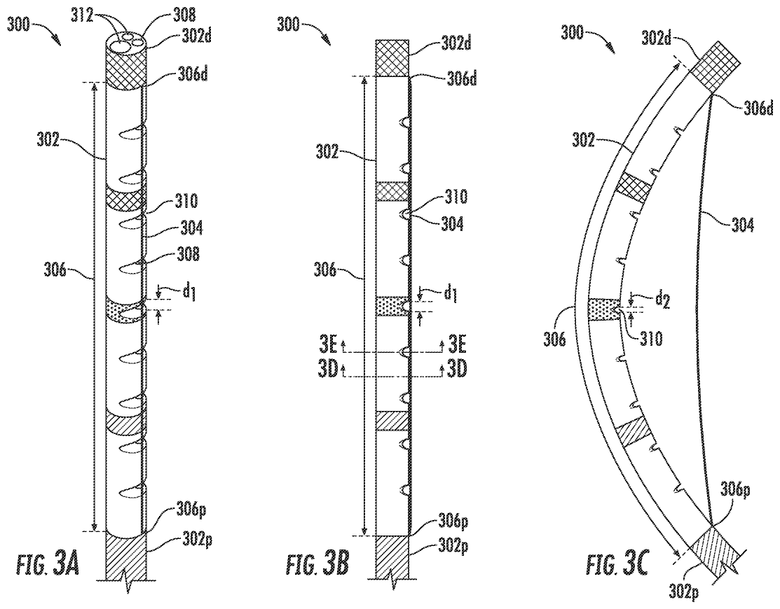

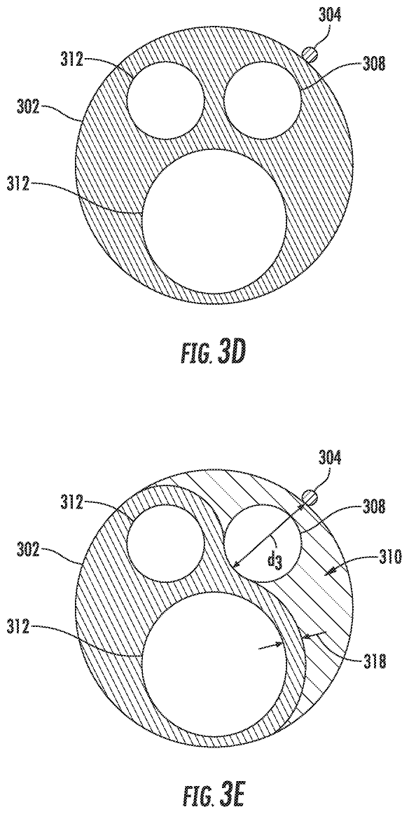

Referring to FIGS. 3A-3E, isometric, right views, and cross-sections of a catheter 300 are illustrated, according to an embodiment of the present disclosure. The catheter 300 includes a shaft 302 extending from a distal end 302d towards a proximal end 302p. The catheter 300 is a multi-lumen catheter 300, including a wire lumen 308 and two accessory lumens 312. Although three lumens 308, 312 are illustrated, in various embodiments, any number of lumens may be employed, e.g., 0, 1, 2, 4, 5, 10, etc. The shaft 302 includes an actuating portion 306. The actuating portion 306 is in a substantially straight configuration in FIGS. 3A and 3B and the actuating portion 306 is arced in the bowed configuration in FIG. 3C. The actuating portion 306 is being pulled by a wire 304 into the bowed configuration of FIG. 3C. The wire 304 extends through a wire lumen 308 along the shaft 302 proximal of the actuating portion 306, and outside of the shaft 302 along the actuating portion 306 from a proximal end 306p to a distal end 306d of the actuating portion 306. The actuating portion 306 may transition between the bowed configuration and the substantially axial configuration by translating the wire 304 through the wire lumen 308 such that tension is applied or released from the distal end 306d of the actuating portion 306. Although the wire lumen 308 is illustrated through the distal end 302d of the shaft 302, the wire lumen 308 and/or one or more accessory lumens 312 may be terminated proximal of the distal end 302d. The wire 304 may also terminate proximal of the distal end 302d of the shaft 302, e.g., a terminal end of the wire 304 may extend into and be anchored in the wire lumen 308 at the distal end 306d of the actuating portion 306.

The actuating portion 306 of the shaft 302 includes slots 310 disposed and spaced-apart from each other along the actuating portion 306. In the substantially axial configuration of FIGS. 3A and 3B, each of the slots 310 have a gapped spacing distance d₁. In the bowed configuration of FIG. 3C, each of the slots 310 have a gapped spacing distance d₂ that is smaller than that of d₁. In the bowed configuration, the actuating portion 306 of the shaft 302 has less compressive force along the shaft 302 (compared to the actuating portion 206 of FIG. 2 without slots) because walls of the slots 310 are flexing towards each other to lessen the gap space distance (i.e., from d₁ to d₂). The gapped spacing of each of the slots 310 allows the actuating portion 306 to transition to the bowed configuration with a substantially uniform arc along the actuating portion 306. Although nine slots 310 are illustrated, in various embodiments, any number of slots may be employed, e.g., 1, 2, 3, 4, 5, 6, 7, 8, 10, 12, 20, 50, etc. As best illustrated in the cross-section of the shaft 302 in FIG. 3E taken along the actuating portion 306 of FIG. 3B, the slots extend radially into (e.g., partially through or across, or entirely through or across, etc.) the wire lumen 308. The arc of the bowed configuration of the actuating portion 306 of FIG. 3C is uniformly distributed along the slots (i.e., the reduced lengths of d₁ to d₂ of each of the slots 310) rather than at a substantially central portion of the arc where a compressed portion of the shaft 302 may kink. A depth d₃ of the slots 310 provides more space for compression (i.e., along the actuating portion 306 in the bowed configuration) compared to slots or etchings with a depth smaller than that of d₃. The depth d₃ of the slots 310 may extend to a lengths e.g., as much as permissible such that a wall of the shaft 302 about the lumens 308, 312 has a thickness t to maintain function of the lumens 308, 312. In various embodiments, exemplary dimensions for slots 310 for illustrative purposes only may be about 1.25 mm for d₁, about 0.75 mm for d₂, about 0.6 mm for d₃, and about 0.075 mm (about 0.003 inches) or about 0.05 mm to about 0.10 mm for t. The wire 304 extends external to the slots 310 at the actuating portion 306 and may rest against the shaft 302 substantially centrally across the slots 310 (e.g., as illustrated in FIGS. 3A, 3B, 3D, and 3E) in an unactuated configuration of the catheter 300. In various embodiments, an axial length of spacing between each of the slots 310 may be longer, similar, or shorter than an axial length of each of the slots 310, and the slots 310 may be uniformly distributed or not uniformly distributed along a length of the actuating portion 306 depending on bowing desires and/or shaft 302 parameters. In various embodiments, exemplary spacing between slots 310 for illustrative purposes only may be between approximately 1 and 2 mm, or about 1.8 mm.

In addition to other functions and benefits of including slots along a shaft of a catheter discussed in this disclosure, slots may additionally reduce the mass of an actuating portion of a shaft to arc into a curve, improving bowing characteristics. Slot cutting or other negative manufacturing may ensure that integrity of the catheter and lumen(s) are not compromised compared to forming shafts of catheters to include the slots, e.g., molding. Slot cutting may be performed as a finishing step in manufacturing, reducing the need for drastic manufacturing strategy shifts in manufacturing shafts of catheters.

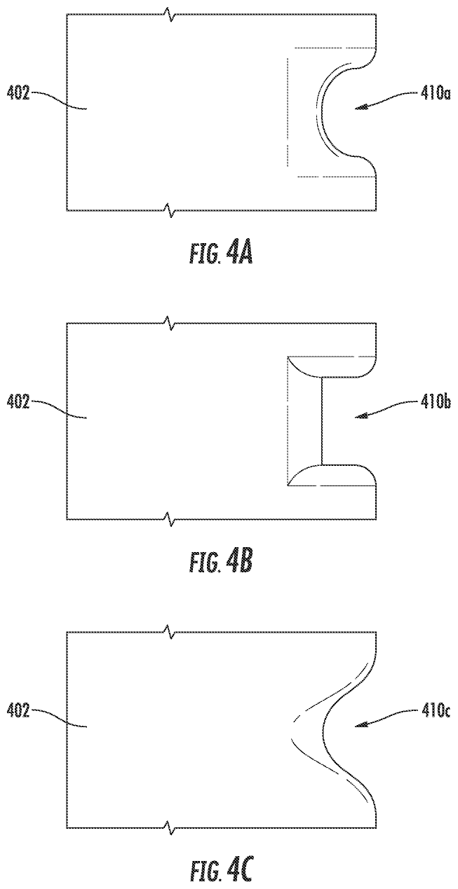

Referring to FIGS. 4A-4C, right views of exemplary shafts 402, each including a slot having a different transverse profile, are illustrated according to embodiments of the present disclosure. Slot geometry may be selected depending on shaft parameters such as number of lumens, size of lumens, length of the shaft, length of the actuating portion, materials of a shaft, desirable arc shape in a bowed configuration, compression forces, compression distances of flexing walls of slots, manufacturability and the like. In FIG. 4A, a slot 410a has a profile that is U-shaped. In FIG. 4B, a slot 410b has a profile that is rectangular. In FIG. 4C, a slot has a profile that is a sloped valley in shape. In various embodiments, a profile of a slot may comprise one or more of the shapes of FIGS. 4A-4C, a V-shape, a slit, a channel, a square, an ellipsoid, a combination thereof, or the like. Embodiments are not limited.

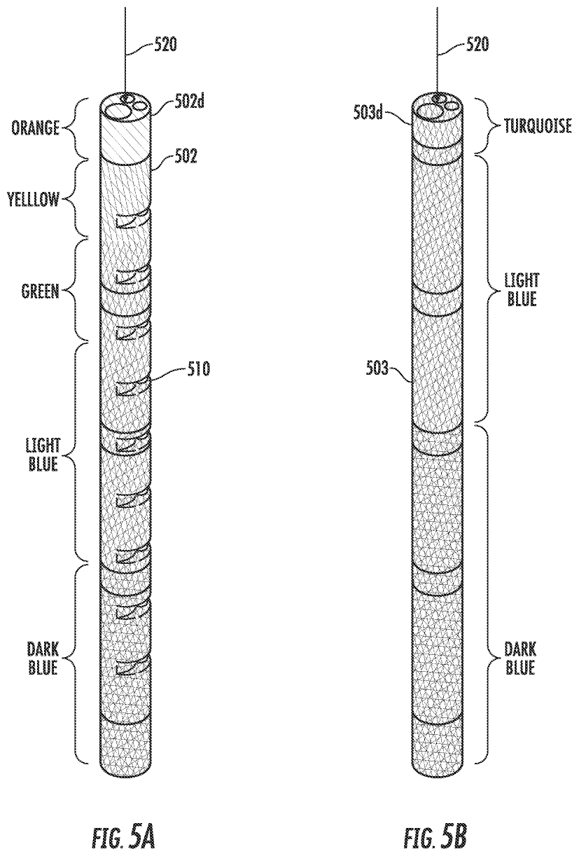

FIGS. 5A and 5B illustrates isometric views of finite element analysis of a slotted shaft 502 and a non-slotted shaft 503, respectively, according to an embodiment of the present disclosure. The shaft 502 includes slots 510 along its length. An axial force vector 520 is being applied to a distal end 502d of the shaft 502. The same axial force vector 520 is being applied to a distal end 503d of the shaft 503 without slots. As illustrated by the shaded and labeled coloring across FIGS. 5A and 5B, the shaft 502 with the slots 510 is deflecting more (ranging from a dark blue through an orange color, i.e., about five colors generally illustrating deflection through color variance), while the shaft 503 without slots is deflecting less (i.e. about three colors illustrating deflection), both under the same axial force 520. In other words, the slotted shaft 502 deflects more than the non-slotted shaft 503 with the same axial force 520. As discussed herein, it may be desirable to achieve more shaft deflection without additional force.

In various embodiments, a distal portion and/or a distal end of a shaft may be articulated via proximal or distal translation of one or more wires. A proximal translation of a wire may pull the distal end of a shaft such that the distal portion of the shaft bows toward the translated wire. A medical professional may translate one or more wires via a handle at the proximal end of the shaft containing a pulley assembly connected to a wire that is actuatable to translate the wire within the wire lumen. In various embodiments, a wire lumen may be filled and/or occluded at an actuating portion of a shaft of a catheter. In various embodiments, a lumen of the shaft may be configured to receive a guidewire such that a distal portion of the guidewire may extend distally beyond the distal end of the shaft. The shaft may include a distal portion configured to move with the translation of a wire (e.g., bow, flex, bend, rotate, wobble, spin, etc.) in a plurality of directions (e.g., x, y, and/or z directions), thereby imparting an identical or similar direction of movement to the distal portion of the guidewire and/or the distal end of the shaft.

In various embodiments, a wire may be conductive and coupled to a power source in order to deliver energy to the wire. The wire may be insulated along at least a portion of a length of the wire and/or the wire may be non-insulated along at least a portion of the length of the wire.

In various embodiments, described here or otherwise within the scope of the present disclosure, a sphincterotomy procedure may be performed by articulating a device, according to one or more embodiments, such that the distal end of the device is against an opening of a body lumen (such as a papilla of vater). The distal end may be advanced through the body lumen and advanced through the lumen and/or toward a desired duct. A guidewire may be advanced through a lumen of the device. The location and position of portions of the device and/or the guidewire may be observed, e.g., via fluoroscopy. Further articulating and advancing of the device and/or the guidewire may be performed to achieve cannulation by bowing the actuating portion the device so that the distal end is oriented toward the opening of the body lumen. A medical professional may articulate a distal end of the device to manipulate and/or cannulate an opening of a body lumen. A medical professional may energize a wire in contact with tissue of the body lumen to assist with opening of the body lumen. The device may be removed from the patient, leaving one or more guidewires in the body lumen to guide another device to be used in the procedure, or the same device used to perform the sphincterotomy may continue to be used for the procedure.

In various embodiments, one or more shafts of a device may comprise an extrusion of multiple lumens. The lumens may be extended through a proximal end of the shaft to the distal end of the shaft, and a portion of the lumen(s) may be exposed to an external surface of the shaft. One or more lumens (e.g., a wire lumen) may terminate at a point that is proximal to the distal end of the shaft. The lumens may be configured to accept instruments and/or fluids (e.g., a contrast agent, a wire, a guidewire) of the device through at least a portion of the lumen and may extend out of a lumen where the lumen is exposed to the outer surface of a shaft. Such instruments may extend partially along a lumen and may extend external to the lumen along various portions of the lumen exposed to an outer surface of the shaft. Such instruments or portions of an instrument extending external to a lumen may be configured to break through a wall of the lumen (e.g., through perforations, thin walls, apertures, or the like) and/or shaft such that the instrument is external to the lumen at the outer surface of the shaft. One or more ends of the shaft may be drawn down to a tapered thinner outer diameter (compared to the remainder of the shaft) such that some or all of the lumens decrease in inner diameter and taper distally to a closed distal end. Additionally, or alternatively, the shaft may be treated at the tip such that some or all of the wire lumens are closed at an end. A drawn-down distal or proximal end may have a smaller diameter than a diameter of other portions of the shaft. A portion of a lumen may be a C-shaped channel extending along a portion of the flexible elongate shaft, e.g., where a portion of a wire may extend outside of the shaft. The channel may comprise other shapes such as, e.g., U-shaped, V-shaped, triangular, boxed, or a combination of these shapes, etc. A channel may be an open channel or a closed channel. A closed channel may have one or more portions of varying wall thickness, e.g., a thinner wall at a portion of the channel.

In various embodiments, the lumens within the shaft may be arranged in different configurations and combinations depending on the instrument and requirements for a particular application. For example, a lumen configured for a guidewire may also be configured (or instead be configured) for delivery of a contrast agent, etc. Embodiments described and illustrated herein are not meant to exclusively include only those lumens, and do not necessarily need to include all of the lumens illustrated.

In various embodiments, fluoroscopy may assist in positioning a system or medical device according to the present disclosure, or for confirming the location of lumens, wires, tissues, presence of one or more bodies such as gallstones, etc. A contrast agent may be injected through a lumen of the medical device and into or about the body lumen for performing fluoroscopy. A guidewire may be withdrawn from the lumen to allow the contrast agent to be injected through the same lumen. Alternatively, a contrast agent may be injected in the lumen containing the guidewire such that the contrast agent flows through the lumen about the guidewire. Alternatively, a lumen or a portion of a multi-lumen shaft (e.g., a bifurcated lumen) may be dedicated to the flow of a contrast agent from a medical device. Alternatively, a lumen may be used for a first purpose and later be used for a second purpose, e.g., first to receive a guidewire and second to flow a contrast agent.

A method of cannulating an opening in a body lumen of the present disclosure may include inserting a catheter into the body lumen of a patient. A wire coupled to a distal end of a catheter may be translated proximally such as via a slider along a handle of the catheter. A portion of the catheter may be curved towards the opening of the body lumen, such as at an actuating portion and/or distal end of a shaft of the catheter. The wire may extend through at least a portion of a first lumen of the catheter. The catheter may comprise a plurality of slots along the actuating portion of the catheter, and the plurality of slots may extend radially into the first lumen. Curving the portion of the catheter may comprise reducing an axial width of each of the plurality of slots. A fluid may be supplied through a second lumen of the catheter into the opening of the body lumen. Proximally translating the wire may comprise pulling the wire toward a handle of the catheter external to the plurality of slots. A portion of the opening of the body lumen may be cut by electrically activating the wire.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A catheter comprising:
a shaft comprising a proximal end, a distal end, an actuating portion proximal to the distal end, and a first lumen extending along a length of the shaft between the proximal and distal ends; and
a plurality of slots disposed and spaced-apart from each other along the actuating portion, the plurality of slots extending radially into the first lumen.

2. The catheter of claim 1, further comprising a wire coupled to the distal end of the shaft and extending externally along the actuating portion of the shaft.

3. The catheter of claim 2, wherein the wire is disposed within the first lumen proximal to the actuating portion.

4. The catheter of claim 2, wherein the wire is disposed substantially at a midpoint across the plurality of slots.

5. The catheter of claim 1, wherein each of the plurality of slots extends radially through the first lumen.

6. The catheter of claim 1, further comprising a second lumen extending through the shaft, wherein the plurality of slots is external to the second lumen.

7. The catheter of claim 6, wherein a wall thickness of the second lumen ranges from about 0.05 mm to about 0.10 mm.

8. The catheter of claim 1, wherein a cross-sectional profile of the plurality of slots transverse to a longitudinal axis along the length of the shaft comprises a U-shape, a V-shape, a box-shape, or a combination thereof.

9. The catheter of claim 1, wherein an axial spacing between each of the plurality of slots along the length of the shaft is longer than an axial length of each of the plurality of slots.

10. The catheter of claim 1, wherein the plurality of slots is uniformly distributed along a length of the actuating portion.

11. The catheter of claim 2, further comprising a handle coupled to a proximal end of the wire, the handle configured to proximally and distally translate the wire through the first lumen.

12. A catheter comprising:
a shaft comprising a proximal end, a distal end, a central longitudinal axis therethrough, an actuating portion proximal to the distal end, and a first lumen extending along a length of the shaft between the proximal and distal ends;
a wire coupled to the distal end, extending external to the shaft along the actuating portion, and within the first lumen proximal of the actuating portion; and
at least one slot disposed along the actuating portion, the slot extending radially into the first lumen, wherein the slot is disposed between the central longitudinal axis and the wire.

13. The catheter of claim 12, wherein the slot extends radially through the first lumen.

14. The catheter of claim 12, further comprising a second lumen extending along the length of the shaft, wherein the slot is external to the second lumen.

15. The catheter of claim 14, wherein a wall thickness of the second lumen ranges from about 0.05 mm to about 0.10 mm.

16. A method of cannulating an opening in a body lumen, comprising:
inserting a catheter into the body lumen of a patient; and
proximally translating a wire coupled to a distal end of a catheter to curve an actuating portion of the catheter towards the opening in the body lumen;
wherein the wire extends through at least a portion of a first lumen of the catheter; and
wherein the catheter comprises a plurality of slots along the actuating portion of the catheter, the plurality of slots extending radially into the first lumen.

17. The method of claim 16, wherein curving the actuating portion of the catheter comprises reducing an axial width of each of the plurality of slots.

18. The method of claim 16, further comprising supplying a fluid through a second lumen of the catheter into the body lumen.

19. The method of claim 16, wherein proximally translating the wire comprises pulling the wire toward a handle of the catheter external to the plurality of slots along the actuating portion.

20. The method of claim 16, further comprising cutting a portion of the body lumen by electrically activating the wire.
